(19) United States
(12) Patent Application Publication
(10) Pub. No.: US 2022/0192343 A1
(43) Pub. Date: Jun. 23, 2022

(54) TRANSPARENT BOTTLE WITH UV COATING FOR UV NAIL GEL COMPOSITIONS, SYSTEMS AND METHODS

(71) Applicant: L'Oreal, Paris (FR)

(72) Inventors: Christopher Pang, Nanuet, NY (US); I-Fan Hsieh, Scotch Plains, NJ (US); Jason Fyfe, Holmdel, NJ (US); Everett Betts, Clark, NJ (US)

(73) Assignee: L'Oreal, Paris (FR)

(21) Appl. No.: 17/491,261

(22) Filed: Sep. 30, 2021

Related U.S. Application Data
(60) Provisional application No. 63/128,990, filed on Dec. 22, 2020.

(30) Foreign Application Priority Data
Feb. 8, 2021 (FR) 2101171

(51) Int. Cl. A45D 34/00; B65D 23/08; B65D 81/30; A61K 8/04; A61Q 3/02

(52) U.S. Cl. CPC A45D 34/00; B65D 23/0814; B65D 81/30; A61K 2800/87; A61Q 3/02; A45D 2200/053; A61K 2800/81; A61K 8/042

(57) ABSTRACT

A UV nail gel and clear bottle system and method for protecting the ultraviolet (UV) nail gel formulation from curing or gelling while providing visibility of the UV nail gel formula. The system includes one or more layer of a transparent UV blocking treatment located on an exterior surface of the clear bottle comprising one or more UV absorber having an intramolecular hydrogen bond and absorption wavelengths ranging from 100 nanometers to 380 nanometers, paired with a UV nail gel formula comprising a lower curing photoinitiator having an absorption wavelength≤330 nm. The one or more layer of the transparent UV blocking treatment and UV nail gel formula with the lower curing photoinitiator protects the UV nail gel formulation from curing or gelling while providing visibility of the UV nail gel formula.

### CROSS-REFERENCES TO RELATED APPLICATIONS

This application claims the benefit of U.S. Provisional Application No. 63/107814, filed Oct. 30, 2020, and French Application No. 2012608, filed Dec. 3, 2020, the disclosures of which are expressly incorporated herein by reference in their entirety.

### SUMMARY

The present disclosure relates to a clear bottle with one or more transparent ultraviolet (UV) coating layers containing a UV nail gel or polish composition having a photoinitiator contemporaneously operable to prevent the UV nail gel formula from curing, gelling, while providing visibility of the UV nail polish.

Typical UV gel nail polish formulations include methacrylate monomers and a photoinitiator, such as benzoyl peroxide. Upon exposure to UV light, the nail polish cures, polymerizing and solidifying. Curing wavelengths typically range between 340 to 380 nanometers (nm). However, when exposed to light over time, the nail polish cures, gelling and developing clumps in its storage vessel or bottle.

Accordingly, UV nail polish gels cannot typically be stored in clear or transparent bottles, and thus a color indicator or description must be used in order to notify the consumer of the color. In order to mitigate or prevent curing of the UV nail polish composition when in the bottle, conventional bottles must be tinted or opaque to block UV light. There is a need in the art for a system providing a clear or transparent bottle containing UV gel nail polish formulations so that the color, tint and pigment complexion of the nail polish is readily visible to a user.

The subject disclosure provides a two-part system. The first part of the system comprises a transparent UV Blocking treatment layers or coated onto a clear flint glass, or clear polymeric, bottle that is able to block UV wavelength from 100 nm-380 nm to prevent the formula from easily be gelled by natural light. The second part of the system comprises a UV nail polish or gel formula that comprises a lower wavelength curing photoinitiator, preferably less than 330 nm, to ensures the compatibility of the nail polish gel formula with the clear UV blocker coated transparent bottle. Combining of the modified UV curable formulation results in stability within the clear UV protective coated glass bottle as compared to conventional coatings.

In an embodiment, a UV nail gel and clear bottle system for protecting the UV nail gel formulation from curing or gelling while providing visibility of the UV nail gel formula is provided. The system includes one or more layers of a transparent UV blocking treatment located on an exterior surface of the clear bottle comprising one or more UV absorbers having an intramolecular hydrogen bond and absorption wavelengths ranging from 100 nm to 380 nm, paired with a UV nail gel formula comprising a lower wavelength curing photoinitiator having an absorption wavelength≤330 nm. The one or more layers of the transparent UV blocking treatment and UV nail gel formula with the lower wavelength curing photoinitiator protects the UV nail gel formulation from curing or gelling while providing visibility of the UV nail gel formula.

In an embodiment, the UV absorbers is selected from o-hydroxybenzophenones, 2-(2-hydroxyaryl)-benzotriazoles and 2-(2-hydroxyaryl)-1,3,5-triazines.

In an embodiment, the photoinitiator is 1-Hydroxycyclohexyl phenyl ketone.

The UV blocking treatment preferably comprises at least two layers on the exterior surface of the clear bottle. More preferably, the UV blocking nail polish and clear bottle system of claim 1, wherein the UV blocking treatment comprises at least three layers on the exterior surface of the clear bottle.

In an embodiment, the one or more layers of UV blocking treatment on the exterior surface of the clear bottle has a thickness ranging between 0.1 um to 200 um.

In yet another embodiment, the clear bottle is a glass bottle. In another embodiment, the clear bottle is a polymeric bottle.

Another embodiment provides that the one or more layers of a transparent UV blocking treatment located on an exterior surface of the clear bottle is applied by spraying.

The one or more layers of a transparent UV blocking treatment located on an exterior surface of the clear bottle may be applied by film wrap.

In an embodiment, the one or more layers of a transparent UV blocking treatment substantially covers the substantially the entire exterior surface of the clear bottle.

Yet another embodiment provides that the one or more layers of a transparent UV blocking treatment substantially covers a bottom portion, body and at least part of a neck portion of the clear bottle. The bottom portion and body representing the main cavity containing the UV nail gel.

A method of making a UV blocking nail polish and clear bottle system is also provided. The method comprises the steps of, in any order, applying one or more layers of a transparent UV blocking treatment to an exterior surface of the clear bottle comprising one or more UV absorbers having an intramolecular hydrogen bond and absorption wavelengths ranging from 100 nm-380 nm; inserting within the clear bottle a UV gel nail polish formula comprising a lower wavelength curing photoinitiator having an absorption wavelength less than 330 nm. Wherein the one or more layers of the transparent UV blocking treatment and UV nail gel formula with the lower wavelength curing photoinitiator protects the UV nail gel formulation from curing or gelling while providing visibility of the UV nail gel formula.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and advantages of the inventive technology will become more readily appreciated as the same become better understood by reference to the following detailed description, when taken in conjunction with the accompanying drawings, wherein:

FIG. 1 is a graph showing wavelength and light transmittance for bottle samples;

FIG. 2a is a photograph showing comparative non-inventive Samples A-0 and A-1 after 24 hours of sun test or light exposure;

FIG. 2b is a photograph showing comparison of non-inventive Sample B-0, and Inventive Samples B-1 to B-3 after 24 hours of light exposure;

FIG. 2c is a photograph of the Samples of FIG. 2b after 48 hours of light exposure.

### DETAILED DESCRIPTION

Conventional UV nail polish bottles are tinted to prevent the UV Nail polish formula from gelling (cures at 340 nm-380 nm) since they can easily cured by light over time. Accordingly, typical UV sensitive formula are stored in a tinted bottle, so the nail composition color is not visible to consumers.

The subject bottle with UV coating for nail polish compositions, systems and methods of use provides a clear (see-through) processing treatment on the outside or exterior of a clear bottle. The clear bottles are treated with clear UV blocker formulation able to block UV wavelength from 100 nm-380 nm. Treatment of the bottles is carried out through spray clear coat, rub, vacuum chamber application, shrink wrap, or other processes or combinations thereof to achieve at least one coating or layer having a thickness to add protection of the formula from UV degradation. In combination, the UV nail gel formula is modified to have a curing wavelength of less than 330 nm to ensure the formula is well protected by the clear coat.

Owing to the pairing of the UV blocking coating with the low wavelength UV curing nail formulation to protect the highly UV sensitive cosmetic formulation while being able to see the color of the formula. As a result, consumers are able to perceive the formulation physical properties, such as color, texture, visual effects, etc., through the clear transparent film, and the UV sensitive formula would still be protected from gelling and/or degradation due to UV light exposure. Preferably, the UV blocking clear transparent film or coating comprises at least one coating, and preferably covers 360 degrees around the bottle. Most preferably the UV clear transparent film covers substantially the entire exterior surface area of the bottle.

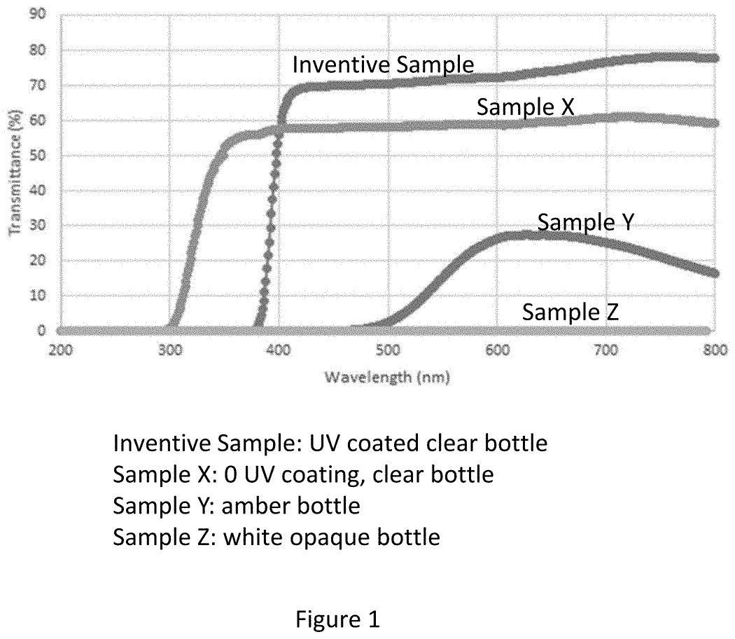

FIG. 1 is a graph showing wavelength and light transmittance for bottle samples. Inventive Sample is a UV coated bottle according to an embodiment. Sample X is a clear, non-UV coated bottle. Sample Y is an amber bottle. Sample Z is a white opaque bottle. Inventive Sample indicates blocking or low transmittance below about 380 nm as compared to Sample X, no UV coating, clear bottle. Sample Y, amber bottle, indicates low transmittance below about 480 nm. Sample Z, the white opaque bottle, resulted in low transmittance across the wavelength spectrum. However, use of white opaque bottle of Sample Z is disadvantageous because there is no visibility of the UV nail polish/gel or treatment. Sample Y, amber bottle, also does not provide visibility of the UV nail polish/gel, but shows a distorted color/indicators of the gel. Comparatively, Inventive Sample indicates increased transmittance % at greater wavelengths, above about 410 nm, demonstrating visibility for viewing the UV nail gel formula in the bottle.

Conventional UV nail polish formula gel or cure at about 340 nm-380 nm. Sample X, 0 coating clear absorbs wavelengths>/=300 nm, generally. Inventive Sample, UV coated clear bottle, transmittance begins at wavelengths of about 380 nm. Inventive Sample is capable of blocking UV wavelength from 100 nm to 380 nm to prevent the UV nail formula from gelling by natural light. Operating contemporaneously with the subject UV gel formula modified with a lower curing photoinitiator, less than 330 nm, ensures the compatibility of formula with the clear UV blocker coating on the transparent bottle. Combining the UV curable formulation shows the best stability with a clear glass coated with UV protective coating.

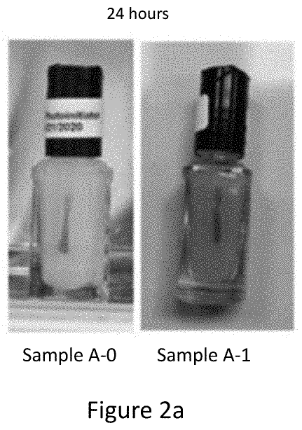

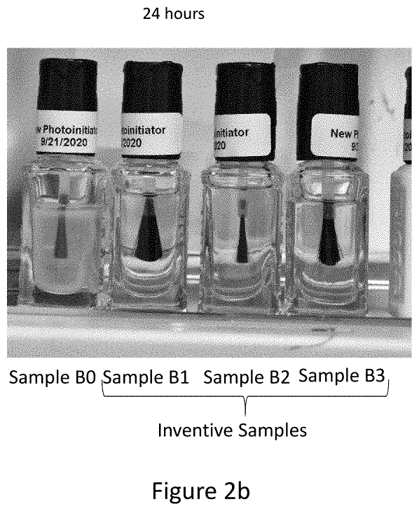

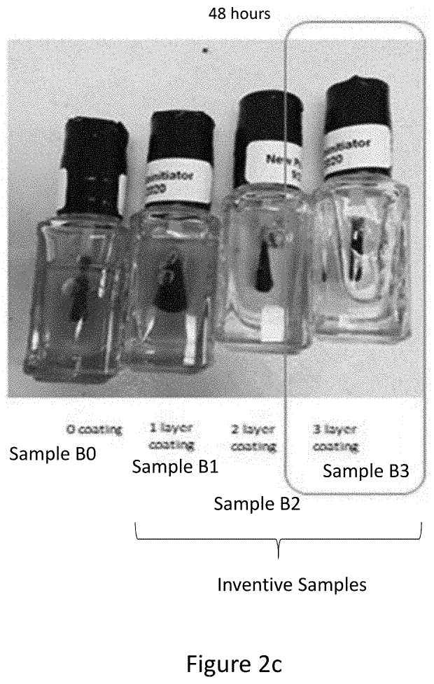

FIG. 2a is a photograph showing comparative non-inventive Samples A-0 and A-1 after 24 hours of sun test or light exposure. FIG. 2b is a photograph showing comparison of non-inventive Sample B-0, and Inventive Samples B-1 to B-3 after 24 hours of light exposure. FIG. 2c is a photograph of the Samples of FIG. 2b after 48 hours of light exposure. Sun test methodology: exposing the samples in a UV light box that is ON constantly.

Referring to FIGS. 2a and 2b, all samples utilized clear glass bottle. Sample A-0, no coating, formula with UV curing range 340 nm-380 nm. Sample A-1, 1-layer coating, formula with UV curing range 340 nm-380 nm. Sample BO represents example with no, or zero (0), UV coating on exterior of bottle, and UV nail formula having UV curing range<330 nm. Inventive Samples B1-B3 represent examples with one (1) layer, UV coating on exterior of bottle—up to 3 coatings or layers, and UV nail formula having UV curing range<330 nm. UV absorbers preferably utilized in the UV coating of the exterior surface of the bottle comprise a UV blocker having one or more intramolecular hydrogen bonds. Preferably, the UV blocker is selected from o-hydroxybenzophenones, 2-(2-hydroxyaryl)-benzotriazoles, 2-(2-hydroxyaryl)-1,3,5-triazines), and combinations thereof.

After 24-hour sun test, both Sample A-0 and A-1 had cured, gelled or solidified completely. Even with the one layer of UV coating on the bottle for Sample A-1, after 24 hour sun test, the UV nail formula solidified. After 48 hours, formula of Sample B3 had the most optimal flow, 100% flow with no curing. Accordingly, 3-layer application of the UV blocking coating in combination with the claimed UV gel formulation with photoinitiator demonstrated the most optimal performance over time.

| | Bottle: # of | UV nail | | |
|---|---|---|---|---|
| | exterior UV | formula: UV | Results: | Results: |
| Sample | coating(s) | curing range | 24 hour | 48 hours |
| Sample A | 0 coating | 340 nm- | cured; | — |
| | | 380 nm | solidified/ | |
| | | | gelled | |
| Sample B0 | 0 coating | ≤330 nm | some | cured; |
| | | | solidification | significant |
| | | | | solidification |
| Inventive | 1 coatings | ≤330 nm | clear; no | very slightly |
| Sample B1 | | | curing | cloudy |
| Inventive | 2 coatings | ≤330 nm | clear; no | very very |
| Sample B2 | | | curing | slightly |
| | | | | cloudy |
| Inventive | 3 coatings | ≤330 nm | clear; no | clear; no |
| Sample B3 | | | curing | curing |

Sun test was carried out, wherein the samples were exposed to sun light and examined for curing state, specifically at 1 hour and 24 hours. Examples above illustrate 1) Sample A with no UV coating and UV nail gel with photoinhibitor at 340 nm-380 nm resulted in cloudy, gelling at 1 hour, and very cloudy, increased gelling at 24 hours. Sample B0 with 0 coating and UV nail gel with ≤330 nm performed better than Sample A UV nail gel with 340 nm-380 nm, however, comparatively Samples B 1-B3 with combination UV coating plus UV nail gel with ≤330 nm all performed better than Sample A and Sample B at 1 hour and 24 hours. Furthermore, of Samples B1-B2, it was found that 3 coatings most preferred, resulting in clear visibility at 1 hour and 24 hours.

### EXAMPLE(S)

Clear glass bottle treated with any UV protective coating. Clear bottles treated with a clear or transparent UV blocker by spray clear coat application. Application of the clear UV blocker can be applied by spray, rub, vacuum chamber, shrink wrap, etc., onto the exterior of the bottle to act in concert with the UV nail gel with photoinhibitor at ≤330 nm for compounded protection of the formula from UV degradation. As set forth above, at least one layer is applied to the exterior surface of the bottle, preferably at least two layers, and most preferably at least three layers are applied to the exterior surface. It is noted that, while “layers” is referred to, thickness of the layer(s) can be modified so that a single layer may have the thickness of two or more layers. Preferably, coating on the bottle has a thickness ranging from 0.1 um to 200 um, including all layers/the thickness sum of the layers.

UV protective coating ingredients: UV absorbers with an intramolecular hydrogen bond (e.g, o-hydroxybenzophenones, 2-(2-hydroxyaryl)-benzotriazoles and 2-(2-hydroxyaryl)-1,3,5-triazines), UV Resistant Clear Acrylic Coating, such as that sold under the trade name Krylon, acetone, n-butyl acetate, propane, butane, xylene, mixed isomers, ethyl 3-Ethoxypropionate, solvent, polyester, additives (TiO2, Silica, etc.). PTFE, PVDF, FEP, and PEEK™. The only plastics found with excellent resistance are the imides, Polyimide (PI) Polyetherimide (PEI).

UV Nail Gel Examples: Invention Example v. Comparative Example

| | Invention | Comparative |
|---|---|---|
| | Example | Example |
| fatty compounds (i.e. | 5.00 | 5.00 |
| glycol, dipropylene | | |
| dibenzoate) | | |
| hydroxycyclohexyl | 0.10 | — |
| phenyl ketone | | |
| ethyl trimethylbenzoyl | — | 0.10 |
| phenylphosphinate | | |
| polymer(s) (i.e. | 23.71 | 23.71 |
| glycerine, copolymers, | | |
| cellulose, etc.) | | |
| solvent (i.e. acetate, | 71.19 | 71.19 |
| ethyl, butyl, etc.) | | |

It was found that Invention Example above with photoinitiator 1-Hydroxycyclohexyl phenyl ketone, in combination with the UV coated bottle, resulted in clear, non-cloudy UV nail polish composition over three days exposure to light. Comparatively, Comparative Example above with ethyl trimethylbenzoyl phenylphosphinate solution resulted in cloudy UV nail polish composition over three days. Accordingly, Invention Example with photoinitiator 1-Hydroxycyclohexyl phenyl ketone with UV transparent film or coating bottle blocking wavelengths 100 nm-380 nm, particularly 360 nm, on exterior of the bottle resulted in improved protection against degradation in light over an extended period of time.

From the foregoing, it will be appreciated that specific embodiments of the technology have been described herein for purposes of illustration, but that various modifications may be made without deviating from the disclosure.

Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology.

Where methods are described, the methods may include more, fewer, or other steps. Additionally, steps may be performed in any suitable order. Accordingly, the disclosure can encompass other embodiments not expressly shown or described herein.

While several embodiments have been illustrated and described, it will be appreciated that various changes can be made therein without departing from the spirit and scope of the inventive technology.

1. A UV nail polish and clear bottle system, comprising:
one or more layers of a transparent ultraviolet (UV) blocking treatment located on an exterior surface of the clear bottle comprising one or more UV absorbers having an intramolecular hydrogen bond and absorption wavelengths ranging from 100 nanometers to 380 nanometers;
a UV nail gel formula comprising a lower curing photoinitiator having an absorption wavelength≤330 nanometers;
wherein the one or more layers of the transparent UV blocking treatment and UV nail gel formula with the lower curing photoinitiator protects the UV nail gel formulation from curing or gelling while providing visibility of the UV nail gel formula.

2. The UV blocking nail polish and clear bottle system of claim 1, wherein the UV absorbers is selected from o-hydroxybenzophenones, 2-(2-hydroxyaryl)-benzotriazoles and 2-(2-hydroxyaryl)-1,3,5-triazines.

3. The UV blocking nail polish and clear bottle system of claim 1, wherein the photoinitiator is 1-Hydroxycyclohexyl phenyl ketone.

4. The UV blocking nail polish and clear bottle system of claim 1, wherein the UV blocking treatment comprises at least two layers on the exterior surface of the clear bottle.

5. The UV blocking nail polish and clear bottle system of claim 1, wherein the UV blocking treatment comprises three layers on the exterior surface of the clear bottle.

6. The UV blocking nail polish and clear bottle system of claim 1, wherein the one or more layer of UV blocking treatment on the exterior surface of the clear bottle has a thickness ranging between 0.1 microns to 200 microns.

7. The UV blocking nail polish and clear bottle system of claim 1, wherein the clear bottle is a glass bottle.

8. The UV blocking nail polish and clear bottle system of claim 1, wherein the clear bottle is a polymeric bottle.

9. The UV blocking nail polish and clear bottle system of claim 1, wherein the one or more layer of a transparent UV blocking treatment located on an exterior surface of the clear bottle is applied by spraying.

10. The UV blocking nail polish and clear bottle system of claim 1, wherein the one or more layer of a transparent UV blocking treatment located on an exterior surface of the clear bottle is applied by film wrap.

11. The UV blocking nail polish and clear bottle system of claim 1, wherein the one or more layer of a transparent UV blocking treatment located on an exterior surface of the clear bottle is applied through one of spray, rub, vacuum chamber application, shrink wrap, or combinations thereof.

12. The UV blocking nail polish and clear bottle system of claim 1, wherein the one or more layer of a transparent UV blocking treatment substantially covers the substantially the entire exterior surface of the clear bottle.

13. The UV blocking nail polish and clear bottle system of claim 1, wherein the one or more layer of a transparent UV blocking treatment substantially covers a bottom portion and body and at least part of a neck portion of the clear bottle.

14. A method of making a UV blocking nail polish and clear bottle system, comprising:
applying one or more layers of a transparent ultraviolet (UV) blocking treatment to an exterior surface of the clear bottle comprising one or more UV absorbers having an intramolecular hydrogen bond and absorption wavelengths ranging from 100 nanometers to 380 nanometers;
inserting within the clear bottle a UV gel nail polish formula comprising a lower curing photoinitiator having an absorption wavelength less than 330 nanometers;
wherein the one or more layer of the transparent UV blocking treatment and UV nail gel formula with the lower curing photoinitiator protects the UV nail gel formulation from curing or gelling while providing visibility of the UV nail gel formula.
